(19) United States
(12) Patent Application Publication
(10) Pub. No.: US 2025/0129017 A1
(43) Pub. Date: Apr. 24, 2025

(54) METHOD FOR PREPARING ISOCYANATES

(71) Applicant: Covestro Deutschland AG, Leverkusen (DE)

(72) Inventors: Sarmilan Santhakumaran, Köln (DE); Lukas Grave, Burscheid (DE); Pascal Scherpian, Moers (DE); Manfred Kobylka, Burscheid (DE); Tevfik Topal Hasan, Leverkusen (DE); Mark Jacob, Monheim (DE); Christian Jens, Köln (DE); Michael Merkel, Düsseldorf (DE); Franz-Josef Niemeyer, Titz (DE); Christian Toffel, Huai Pong (TH)

(21) Appl. No.: 18/834,257

(22) PCT Filed: Feb. 3, 2023
(86) PCT No.: PCT/EP2023/052656

§ 371 Date: Jul. 30, 2024

(30) Foreign Application Priority Data
Feb. 10, 2022 (EP) 22156144.2
Feb. 10, 2022 (EP) 22156145.9
Sep. 29, 2022 (EP) 22198750.6

(51) Int. Cl. C07C 263/10; C07C 263/20

(52) U.S. Cl. CPC C07C 263/10; C07C 263/20

(57) ABSTRACT

The invention relates to a method for preparing isocyanates by reacting corresponding amines with phosgene. The invention further relates to an apparatus for separating exhaust gas flows from a phosgenation reaction.

The invention relates to a process for producing isocyanates by reacting the corresponding amines with phosgene. In addition, the invention relates to a device for separating offgas streams from a phosgenation reaction.

Isocyanates are important raw materials in the chemical industry. They are often produced in large amounts. Di- and polyisocyanates in particular are used primarily as starting materials for the production of polyurethanes. Their large-scale production is usually accomplished in good yields by phosgenation of the corresponding amines, amine hydrochlorides or amine carbonates with an excess of phosgene. In this reaction, the corresponding carbamoyl chloride is formed as an intermediate, which is then converted into the isocyanate with the elimination of hydrogen chloride.

The solvents for the reaction that have become established in industry are chlorobenzene or o-dichlorobenzene, which are largely inert in their behavior and highly suitable for the recovery of excess phosgene and separation thereof from hydrogen chloride. However, it is also possible to use other solvents that are inert under the reaction conditions.

The process offgas, which consists essentially of phosgene, hydrogen chloride and solvent, must be treated to avoid the release of phosgene. To improve the economic efficiency of the processes, it is also expedient to process the process offgas, which consists essentially of phosgene, hydrogen chloride and solvent, so that at least a portion of these constituents, in particular the phosgene, can be used further or reused. Numerous variants are known for the separation of this process offgas stream, which differ from one another according to the primary objective.

US2007/0249859A1 describes an at least 2-step sequence of absorption steps, the sequence comprising at least one isothermal absorption step and at least one adiabatic absorption step, with the phosgene thus obtained returned to the phosgenation reaction.

In DE102008009761A1, the process offgas stream initially undergoes partial condensation, with the liquid condensate processed in a stripping column. This process affords a liquid solvent stream in the bottoms and a gas stream at the head of the column that is directed into an absorption together with the hitherto uncondensed fractions. The phosgene present therein is absorbed in a solvent and the resulting phosgene solution is reused in the phosgenation reaction, optionally after enrichment with further phosgene.

US2018/0044179A1 is based on a distillative separation of the process offgas. In this case, the offgas stream is directed into a distillation column and a phosgene-containing stream withdrawn from the bottom thereof. At the head of the column, a stream consisting essentially of hydrogen chloride is withdrawn, compressed and so partially condensed. The condensed fraction is depressurized and fed back at the head of the column.

While phosgene is thus often recycled into the same process, there are other options for utilizing the hydrogen chloride. For example, it can be used or marketed simply as an aqueous solution, i.e. as hydrochloric acid. Alternatively, it can be catalytically or electrochemically oxidized to chlorine or used in the oxychlorination of ethylene to ethylene dichloride.

Many isocyanates are produced in large amounts and continuous processes are preferred for this. Isocyanates that are produced in smaller amounts are usually produced by batchwise liquid-phase phosgenation, since the costs involved in switching to a continuous process are very high and are not recoverable given the small amounts to be produced. In some cases, hybrid processes are also operated in which a batchwise reaction regime is combined with continuously operated downstream processing of the isocyanates produced. This requires the installation of buffer containers into which the reaction products are discharged and from which the continuous processing is then supplied.

For batchwise production of isocyanates too, treatment of the offgas and—where possible—recovery of phosgene from this offgas is also desirable. There are however problems with this that have yet to be addressed in the prior art. For example, the process offgas mass flows and composition thereof vary greatly depending on the operating conditions and, while it is relatively easy to install a buffer tank for liquid product streams, thus ensuring an even inflow stream for processing, this is not readily achievable for the process offgas on account of the large volumes and the toxic and corrosive constituents.

U.S. Pat. No. 4,233,267A describes a system combining a batch reactor and a continuous distillation apparatus, wherein the volatile reaction products are first condensed and temporarily stored in a condensate container, from which they are fed into the continuously operated column essentially independently of the operating parameters of the batch reactor. For phosgenation processes, this approach has various disadvantages. Firstly, the condensation of phosgene, let alone HCl, requires very low temperatures. Secondly, the buffering that is necessary means that such an approach inevitably increases the amount of phosgene in the system.

For the operation of an absorption for separating phosgene from a process offgas from isocyanate production, the disclosures of DE102008009761A1 and US2007/0249859A1 both recommend adjusting the amount of solvent for absorption in line with the phosgene mass flow. In said disclosures, the weight ratio of solvent to phosgene at the inlet to the isothermal absorption is preferably in the range from 0.1:1 to 10:1 and more preferably in the range from 1:1 to 3:1.

However, it has surprisingly now been found that the phosgene mass flow to the absorption device is not a good guide for the required or even optimal mass flow of fresh solvent to the absorption device. This is the case particularly in the batchwise production of isocyanates by phosgenation of the corresponding amines, in which there are strong fluctuations in the composition and in the overall mass flow of the process offgas. In fact, it has been observed that, even when the preferred ranges for the solvent-to-phosgene ratio at the inlet to the isothermal absorption are maintained, phases of phosgene penetration into the offgas of the absorption device occur, while in other phases an unnecessarily large amount of solvent has been expended.

It was thus an object of the invention to provide an improved process for producing isocyanates and also a device for separating offgas streams from a phosgenation reaction with which, despite fluctuations in the composition and mass flow of the process offgas from a phosgenation, phosgene can be separated from the process offgas by absorption in a reliable and resource-conserving manner.

This object was achieved by a process for producing an isocyanate, comprising the steps of:
A) reacting at least one amine or salt thereof with phosgene, affording a liquid reaction product comprising the isocyanate and also a first gaseous process offgas stream comprising hydrogen chloride and phosgene,
B) separating the gaseous first process offgas stream comprising hydrogen chloride, phosgene, any solvent, any low-boiling secondary components and any inert constituents in at least one absorption device, in which the first process offgas stream is contacted with an absorbent, affording a phosgene-depleted second offgas stream having a first phosgene content and a first phosgene solution having a second phosgene content,
C) optionally mixing the first phosgene solution obtained in step B) with additional phosgene and/or solvent to obtain a conditioned second phosgene solution,
D) optionally using the first phosgene solution from step B) or the conditioned second phosgene solution from step C) for phosgenation of an amine,
E) optionally isolating the isocyanate by distillative separation of the liquid reaction product obtained in step A,
characterized in that
the first phosgene content of the phosgene-depleted second offgas stream obtained in step B) is compared against a first target value for said first phosgene content and/or the second phosgene content of the first phosgene solution obtained in step B) is determined and compared against a predefined second target value for said second phosgene content, and in that
the absorbent having a mass flow M1 is introduced into the absorption device and this mass flow M1 is under closed-loop control, wherein the reference variable for the mass flow M1 is altered, preferably automatically altered, when the first phosgene content and/or the second phosgene content deviate from their respective target value.

According to the invention the terms “comprising” or “containing” preferably mean “consisting essentially of” and more preferably mean “consisting of”. The further embodiments recited in the claims and in the description may be combined as desired, provided that the context does not clearly indicate the opposite.

“At least one”, as used herein, refers to 1 or more, for example 2, 3, 4, 5, 6, 7, 8, 9 or more. In connection with constituents of the compounds described herein, this figure refers not to the absolute number of molecules, but rather to the nature of the constituent. “At least one amine” therefore means for example that only one type of amine or a plurality of different types of amines may be present without specifying the amount of the individual compounds.

Numerical values specified herein without decimal places refer in each case to the full value specified to one decimal place. For example “99%” signifies “99.0%”.

Numerical ranges given in the format “in/from x to y” include the values stated. If two or more preferred numerical ranges are given in this format, it is understood that all ranges arising from the combination of the various limits are likewise encompassed.

In the present case, “low-boiling secondary components” are compounds that at 10⁵ Pa have a lower boiling point than the isocyanate produced in step A). These can be, for example, chlorinated organic compounds or fragments of the isocyanate produced in step A).

In a preferred embodiment of the process of the invention, the target value for the first phosgene content is in the range from 0.00% by volume to 2.00% by volume, preferably in the range from 0.01% by volume to 1.00% by volume, more preferably in the range from 0.02% by volume to 0.50% by volume, and most preferably in the range from 0.02% by volume to 0.2% by volume. A defined first target value in this range can be compared against a measured actual value for the first phosgene content of the phosgene-depleted second offgas stream obtained in step B). Based on the variance between the target value and actual value, the mass flow M1 of the absorbent into the absorption device can then preferably be adjusted automatically.

In a further preferred embodiment of the process of the invention, the target value for the second phosgene content is in the range from 20% by weight to 70% by weight, preferably in the range from 30% by weight to 68% by weight, and more preferably in the range from 42% by weight to 66% by weight. A defined second target value in this range can be compared against a measured actual value for the second phosgene content of the first phosgene solution obtained in step B). Based on the variance between the target value and actual value, the mass flow M1 of the absorbent into the absorption device can then preferably be adjusted automatically.

In a further preferred embodiment of the process of the invention, the two embodiments described above are combined with one another. In normal operation, the second phosgene content is controlled in this embodiment by adjusting the mass flow M1 to the predefined second target value and, if the first target value for the first phosgene content now serving as the threshold value is exceeded, that is to say there is a deviation from normal operation, namely the penetration of an increased amount of phosgene into the second offgas stream, the mass flow M1 is increased such that more absorbent is introduced into the absorption device than would be required for adjustment of the second phosgene content in the first phosgene solution. The increase in the mass flow M1 here preferably becomes progressively greater according to the extent to which the first target value is exceeded and remains active either for a certain time or preferably until the measured actual value for the first phosgene content has fallen back below the first target value. This is what is known as an override concept, in which the closed-loop control relating to the second phosgene content is inactive for the duration of the phosgene penetration and the mass flow M1 is instead specified on the basis of the first phosgene content. In this way, the desired second phosgene content of the first phosgene solution can in normal operation be maintained as exactly as possible, while at the same time reducing or preventing phosgene penetration into the second offgas stream.

Examples of preferred diamines are diaminotoluene (TDA), especially 2,4-diaminoluene and 2,6-diaminotoluene, diaminodimethylbenzene, diaminonaphthalene, especially 1,5-diaminonaphthalene (NDA), diaminobenzene, especially 1,4-diaminobenzene (pPDI), diaminodiphenylmethane (MDA), especially 2,2′-diaminodiphenylmethane, 2,4′-diaminodiphenylmethane and 4,4′-diaminodiphenylmethane, 1,4-diaminobutane, 1,5-diaminopentane (PDA), 1,6-diaminohexane (HDA), 1,11-diaminoundecane, 1-amino-3,5,5-trimethyl-5-aminomethylcyclohexane (IPDA), bis(p-aminocyclohexyl)methane (PACM), 1,5-diamino-2-methylpentane, 2,5-diamino-2,5-dimethylhexane, 1,4-diaminocyclohexane, hexahydrotolylenediamine (H6TDA), especially 2,4-hexahydrotolylenediamine, 2,6-hexahydrotolylenediamine, 1,3-bis(aminomethyl)benzene (m-XDA), 1,4-bis(aminomethyl)benzene (p-XDA), bis(aminomethyl)cyclohexane (H6-XDA), tetramethylxylylenediamine (TMXDA), bis(aminomethyl)norbornane (NBDA), neopentanediamine, 2,4,4-trimethylhexamethylenediamine, 2,2,4-trimethylhexamethylendiamine, and mixtures thereof.

Examples of particularly preferred diamines are 1,5-diaminonaphthalene (NDA), 1,4-diaminobenzene (pPDA), 1,5-diaminopentane (PDA), 1,6-diaminohexane (HDA), 1-amino-3,5,5-trimethyl-5-aminomethylcyclohexane (IPDA), bis(p-aminocyclohexyl)methane (PACM), 1,5-diamino-2-methylpentane, 2,5-diamino-2,5-dimethylhexane, 1,4-diaminocyclohexane, hexahydrotolylenediamine (H6TDA), especially 2,4-hexahydrotolylenediamine and 2,6-hexahydrotolylenediamine, 1,3-bis(aminomethyl)benzene (m-XDA), 1,4-bis(aminomethyl)benzene (p-XDA), bis(aminomethyl)cyclohexane (H6-XDA), bis(aminomethyl)norbornane (NBDA), and mixtures thereof.

Examples of very particularly preferred diamines are 1,5-diaminonaphthalene (NDA), 1,4-diaminobenzene (pPDA), 1,5-diaminopentane (PDA), bis(p-aminocyclohexyl)methane (PACM), hexahydrotolylenediamine (H6TDA), especially 2,4-hexahydrotolylenediamine and 2,6-hexahydrotolylenediamine, 1,3-bis(aminomethyl)benzene (m-XDA), bis(aminomethyl)norbornane (NBDA), and mixtures thereof.

Examples of most preferred diamines are 1,5-diaminonaphthalene (NDA), 1,4-diaminobenzene (pPDA), bis(p-aminocyclohexyl)methane (PACM), 1,3-bis(aminomethyl)benzene (m-XDA), bis(aminomethyl)norbornane, and mixtures thereof.

Instead of the amines, it is also possible to phosgenate the salts thereof, especially the hydrochlorides or carbamates, preferably the hydrochlorides, of the amines. These salts are in that case usually produced in situ in a first step at low temperature, as described for example in DE19510259A1, it being optionally possible to dispense with the introduction of the inert gas. It is preferable to phosgenate the amines directly (base phosgenation), specifically in a two-stage reaction at different reaction temperatures (cold-hot phosgenation).

The reaction can be carried out in the presence of a solvent. In the case of a gas-phase phosgenation, the presence of a solvent is often dispensed with during the reaction itself; the solvent is instead added only after the reaction, in order to swiftly cool the reaction products. This process is also referred to as quenching. All solvents known to those skilled in the art that are inert toward the prevailing reaction conditions are suitable. Preferred solvents are selected from the group consisting of aromatic hydrocarbons, halogenated aromatic hydrocarbons, especially chlorinated aromatic hydrocarbons, esters, ethers, halogenated hydrocarbons, and mixtures thereof. Aromatic hydrocarbons used with particular preference according to the invention are selected from the group consisting of toluene, bromobenzene, chlorobenzene, dichlorobenzene, especially o-dichlorobenzene, and mixtures thereof. According to the invention, particular preference is given to using chlorobenzene, o-dichlorobenzene or mixtures of these two solvents.

The reaction is preferably carried out in the condensed phase, i.e. as what is known as a liquid-phase phosgenation, in which it is possible also for solids and gases to be present in the essentially liquid reaction mixture.

The process of the invention is particularly advantageous in the production of isocyanates for industrial processes in which the reaction of the at least one amine or amine salt with phosgene is carried out in a batchwise manner. The batchwise production of isocyanates, also known as batchwise or semi-batchwise operating mode, is characterized by particularly significant fluctuations both in the overall mass flow and in the composition of the process offgas.

In a further embodiment, the reaction is carried out in a batchwise manner in the condensed phase. Batchwise reaction is in the present invention to be understood as meaning that the liquid reaction product is not continuously withdrawn from the reactor or reactors, but that the liquid reaction product is withdrawn from a reactor, and transferred to a holding vessel for the crude product or sent directly for further processing, essentially only once conversion into the isocyanate in said reactor is complete. Those skilled in the art are familiar with various methods for determining the end point of the reaction. It is usually what is known as the “clear point” that is used, this being the point at which a clear solution has formed from the suspension initially present in the hot phosgenation. As an alternative, it is also possible to use the end of evolution of HCl gas or the presence of a constant NCO content in the reaction mixture. Even during the reaction, it is however possible to withdraw a portion of the liquid reaction mixture from the reactor and return it back thereto, for example in order to bring about better mixing or to control the temperature of the reaction mixture without contravening the condition of batchwise phosgenation in the context of the present invention. Gaseous flows comprising essentially hydrogen chloride, phosgene, inert gas and solvent vapors may however be withdrawn from the reactors and reprocessed at any time, including continuously. Preferably, the reaction is carried out in what is known as a semi-batchwise process, also referred to as fed-batch process or feed process, that is to say the initially partially filled reactor is fed during the reaction with amine and/or phosgene and optionally inert gas.

Stirred-tank reactors are particularly suitable as reactors, but other types of reactors, such as loop reactors, can also be used in principle. The reaction is preferably carried out at a pressure of from 1.0 bar(a) to 20 bar(a), preferably 1.05 bar(a) to 10 bar(a), and more preferably from 1.1 bar(a) to 6 bar(a). Accordingly, the at least one reactor is preferably equipped with a pressure-retaining device, for example a control valve, via which process offgas can escape from the reactor. In the case of continuous reaction regime, tubular reactors in particular are especially suitable.

In a further preferred embodiment of the process of the invention, the reaction is carried out in the form of a base phosgenation. In a base phosgenation, that is to say the reaction of the amines with phosgene, the reaction is preferably carried out in two stages in the inert solvent. Such reactions are described for example in W. Siefken, Liebigs Annalen der Chemie, 562 (1949) p. 96. In the first stage, the cold phosgenation, the temperature of the reaction mixture is preferably kept within a range between 0 and 100° C. A suspension comprising carbamoyl chloride, amine hydrochloride, and small amounts of free isocyanate forms. The preferred procedure here is for a solution of phosgene in an inert solvent to be initially charged and a solution or suspension of the amine in the same solvent and optionally further phosgene then added. This keeps the concentration of free amine low and thus suppresses the undesired formation of ureas.

In the second stage, the hot phosgenation, the temperature is increased and is then preferably within a range of from 120° C. to 200° C. It is kept within this range during the time in which further phosgene is supplied until the reaction to the isocyanate has ended, i.e. until the evolution of HCl ceases and/or the reaction mixture becomes clear. Phosgene is advantageously used in excess. If required, the reaction may be carried out with introduction of an inert gas both in the cold phosgenation and in the hot phosgenation.

If the amines to be converted are high-melting amines that are sparingly soluble in the solvent, it is also possible to use a suspension of the amine for the phosgenation. This is preferably produced by dispersion with a dynamic mixing unit, as described in EP2897933B1, paragraph [0020]to paragraph [0024].

In the amine hydrochloride or carbamate phosgenation, the amine is preferably initially reacted with hydrogen chloride gas or carbon dioxide in an inert liquid medium to produce the corresponding salt. The reaction temperature during this salt formation is preferably within a range of from 0 to 80° C. An initial reaction with phosgene can already take place subsequently or at the same time. This is followed by a further phosgenation step that is essentially similar to the hot phosgenation from the base phosgenation described above and is therefore likewise referred to hereinafter as hot phosgenation. Here too, the temperature is thus preferably kept within the range from 120 to 200° C. while phosgene and optionally an inert gas are being introduced into the reaction mixture. The introduction is continued until the reaction to the isocyanate has ended. Here too, phosgene is preferably employed in excess in order to speed up the reaction.

The phosgenation is usually carried out with an excess of phosgene, that is to say more than one mole of phosgene per mole of amino groups is used. The molar ratio of total phosgene used to amino groups is preferably 1.02:1 to 20.0:1, more preferably 1.1:1 to 10.0:1, and most preferably 1.2:1 to 5.0:1. If necessary, further phosgene or phosgene solution can be supplied to the reaction mixture during the reaction in order to maintain a sufficient excess of phosgene or to compensate for loss of phosgene.

Both in the base phosgenation and in the phosgenation of the amine hydrochloride or carbamate, the residual phosgene and hydrogen chloride gas is in most cases at least partially removed at the end of the reaction, preferably by purging with an inert gas, preferably with nitrogen, or, less preferably, by removal under reduced pressure. If necessary, the reaction mixture may be filtered to remove any solids present such as unreacted amine hydrochlorides.

In the course of batchwise phosgenations, two maxima normally occur in the mass flow of process offgas for each batch. An increase in the mass flow of process offgas from a reactor occurs for example during the transition to the hot phosgenation. The heating of the reaction mixture lowers the solubility of gases in the mixture, resulting in outgassing of dissolved gases and a consequent increase in the off gas stream from the reactor, which, depending on the selected process, comprises phosgene, hydrogen chloride and/or carbon dioxide in particular. A second maximum occurs at the end of the reaction when residual phosgene and hydrogen chloride are expelled from the reaction mixture and—if the reaction has been carried out under pressure—the reaction vessel is depressurized.

In order to reduce the occurrence of these fluctuations in the process offgas stream in the batchwise process and to permit a more economical design of the apparatuses and operating conditions, it is preferable for the reaction in step A) to be carried out in at least 2 reactors arranged in parallel, more preferably in 2 to 10 reactors arranged in parallel, particularly preferably in 2 to 6 reactors arranged in parallel, and most preferably in 2 or 4 reactors arranged in parallel, where at least one reactor is operated asynchronously to at least one of the other reactors. This means that the heating of the reaction mixture to above 100° C. and/or the expulsion of residual phosgene and hydrogen chloride at the end of the reaction, which also optionally includes the depressurization of the reactor to a lower pressure, are not carried out in all reactors at the same time or under the same conditions.

It is for example possible during the transition to the hot phosgenation to execute the same temperature ramp with a time delay or else, in the case of the reactor not operated synchronously with the other reactors, to execute a different temperature ramp, especially a flatter one, so that the associated maximum in the offgas mass flow is lower and/or does not coincide with the maximum in the offgas mass flows of the other reactors. Particularly preferably, the reactor executes the same temperature ramp with a time delay, such that the temperature increase in the transition to the hot phosgenation occurs either before or after the temperature increase in the other reactors. Preferably, the time delay is at least 5 minutes, more preferably at least 10 minutes, and most preferably at least 20 minutes. Likewise preferably, the time delay is not more than 24 h, preferably not more than 8 hours, more preferably not more than 2 h, and most preferably not more than 1 h.

The same also holds for the expulsion of residual phosgene and hydrogen chloride. In this case, it is for example possible for the depressurization and/or optional introduction of inert gas to be carried out asynchronously in at least two of the reactors arranged in parallel, i.e. with a time delay and/or at different rates, in order in turn to ensure that the associated maximum in the offgas mass flow is lower and/or does not coincide with the maximum in the offgas mass flows in the other reactors. Particularly preferably, the depressurization and/or optional introduction of inert gas is carried out with the same specifications regarding the pressure profiles and inert gas mass flows over time as for the other reactors, but with a time delay. Preferably, the expulsion of phosgene and/or hydrogen chloride is carried out with a time delay of at least 5 minutes, more preferably at least 10 minutes, and most preferably at least 20 minutes. Likewise preferably, the time delay is not more than 24 h, preferably not more than 8 hours, more preferably not more than 2 h, and most preferably not more than 1 h.

In a further preferred embodiment of the invention, the asynchronously operated reactors are operated according to the same operating instructions, but the individual process steps are run through with a time delay of at least 5 minutes, preferably at least 10 minutes, and more preferably at least 20 minutes. It does not matter in which direction the time delay occurs, i.e. which reactor is being operated behind or ahead of the others.

In a further preferred embodiment, none of the reactors arranged in parallel is operated synchronously with any of the other reactors. Preferably, all reactors are operated according to the same operating instructions, but with the individual process steps being run through with a time delay relative to one another.

The first process offgas normally comprises phosgene, hydrogen chloride, and any solvent vapors, inert gas and traces of other volatile components of the reaction mixture, such as amines or isocyanates. This process offgas is separated in step B) in at least one absorption device into a phosgene-depleted second offgas stream and a phosgene-containing absorbent, i.e. a first phosgene solution. Preferably, the separation in step B) is carried out in exactly one absorption device.

Suitable absorption devices are for example gas scrubbers in which the first process offgas stream is contacted with a liquid stream, also termed absorbent or scrubbing medium, examples being immersion scrubbers, spray scrubbers, packed columns with a random packing, packed columns with a structured packing, tray columns or falling-film absorbers. For efficient absorption of phosgene from the first process offgas stream it is also possible to combine different types of gas scrubber. Preference here is given to combining two or more, more preferably two, gas scrubbers in an absorption device. In a further preferred embodiment, the absorption device comprises a gas scrubber selected from the group consisting of packed columns with a random packing, packed columns with a structured packing, and tray columns, preferably consisting of packed columns with a random packing and packed columns with a structured packing.

A particularly suitable absorption device comprises a falling-film absorber that is suitable for carrying out an isothermal absorption and a tray column, packed column with a random packing or packed column with a structured packing, preferably a packed column with a random packing or packed column with a structured packing, more preferably a packed column with a structured packing, that is suitable for carrying out an adiabatic absorption. The falling-film absorber is preferably a vertically arranged shell-and-tube heat exchanger in which the process offgas and the absorbent are conducted either on the tube side or on the shell side of the heat exchanger, preferably on the tube side of the heat exchanger, while on the other side in each case a cooling medium is conducted in cocurrent or countercurrent, preferably in countercurrent, to the process offgas stream. The process offgas and the absorbent can equally be conducted in cocurrent or countercurrent to one another, preferably in countercurrent to one another. The tubes can optionally be furnished with a random or structured packing or other internals to improve mass transfer and/or heat exchange. The tray column, packed column with a random packing or packed column with a structured packing is preferably set up such that the process offgas from the isothermal gas scrubber is contacted in countercurrent with the absorbent. The absorbent is preferably introduced at the upper end of the column via a liquid distributor. The packed column is preferably one with a structured packing.

Particularly preferably, the falling-film absorber and the tray column, packed column with a random packing or packed column with a structured packing are combined in a column-form apparatus that includes at the lower end a supply line for the process offgas. The falling-film absorber is arranged thereabove, and above that the tray column, packed column with a random packing or packed column with a structured packing. In order that the absorbent is distributed evenly on the tubes/on the column, liquid distributors are preferably present above the falling-film absorber and at the head of the column-form apparatus. Optionally present at the head of the absorption device is an internal or external condenser for further cooling of the offgas stream that is now depleted of phosgene.

The second offgas stream, which is as phosgene-depleted as possible, usually consists essentially, i.e. to an extent of at least 80% by volume, preferably to an extent of at least 95% by volume, and more preferably to an extent of at least 98% by volume, of hydrogen chloride. It also contains residual solvent, inert gases if used, and traces of phosgene. The phosgene content of this offgas stream is preferably not more than 1.0% by volume, more preferably not more than 0.5% by volume, and most preferably not more than 0.2% by volume. The natural and generally desirable lower limit for the phosgene content in this offgas stream is 0.00% by volume. However, not only can adherence to this limit lead to increased consumption of absorbent, it may also be advantageous for the design of a closed-loop control unit to permit a low phosgene content in the offgas, for example in order to use this as a closed-loop control variable. Therefore, the phosgene content in the second offgas stream is preferably at least 0.01% by volume, more preferably at least 0.02% by volume, and most preferably at least 0.05% by volume.

In order to achieve maximum efficiency of absorption, it is advantageous to cool the first process offgas initially to a temperature in the range from 5° C. to −25° C., preferably in the range from 0° C. to −20° C., and more preferably in the range from −5° C. to −15° C. The cooling can be effected simply by means of one or more heat exchangers or by quenching, i.e. by contacting the first process offgas with an already cooled liquid in a quench device. This can be done, for example, in a gas scrubber, preferably a spray scrubber, or in an irrigated heat exchanger. A suitable cooled liquid is for example the absorbent. Preferably, the absorbent here, after contact with the process offgas, is deposited as a liquid and this liquid is recirculated into the quench device, where the liquid loop or the quench device itself contains a cooler to cool the liquid to the desired temperature. During cooling, partial condensation/absorption of phosgene occurs.

The residual gas phase is then contacted with the absorbent, preferably in countercurrent. At the same time, the rising gas phase is preferably contacted in countercurrent with the descending liquid absorbent. Suitable devices for this purpose are known to those skilled in the art. Preferably, the absorption comprises a combination of two absorption steps, the gas preferably first passing through an isothermal absorption and then an adiabatic absorption. The volume flow of the absorbent is variable and can in each case be adapted to the prevailing process conditions and the resulting requirements. The absorbent is preferably selected from the group consisting of aromatic hydrocarbons, halogenated aromatic hydrocarbons, especially chlorinated aromatic hydrocarbons, esters, ethers, and halogenated hydrocarbons, or mixtures thereof. According to the invention, particular preference is given to using as absorbent chlorobenzene, o-dichlorobenzene or mixtures of these two solvents. Very particularly preferably, the absorbent is chlorobenzene.

In a further preferred embodiment, a solvent employed as absorbent in the at least one absorption step is preferably the solvent that is also present in the reaction of the at least one amine or salt thereof. In the preferred embodiment with two absorption steps, fresh or reprocessed absorbent is preferably introduced in the second absorption step, i.e. the absorption step that the gas passes through later, whereas in the first absorption step preference is given to using already-laden absorbent from the second absorption step, optionally together with fresh absorbent and/or a recirculated, likewise already-laden absorbent from the first absorption step and/or the preceding cooling process. Preferably, the gas stream after contact with the absorbent passes through a condenser in order to condense absorbed absorbent and to separate it as completely as possible from the gas stream.

The laden absorbent can, as previously described, be employed as a quench medium in order to initially cool the first process offgas stream. A portion of the absorbent is withdrawn either continuously or discontinuously and, optionally after enrichment with further phosgene, used in one of the reactors for phosgenation of the amine or salt thereof. The withdrawn, laden absorbent is preferably a solution containing 20% by weight to 70% by weight, more preferably 30% by weight to 68% by weight, and most preferably 42% by weight to 66% by weight, of phosgene in chlorobenzene. The content of the solution here depends in particular on the phosgene content of the first process offgas, on the amount of fresh absorbent for absorption, and on the amount of absorbent withdrawn.

The closed-loop control systems in the device that have been described according to the invention can be implemented for example with the aid of a computer. Therefore, the invention further provides a device for separating offgas streams from a phosgenation reaction, comprising at least one, preferably exactly one, absorption device for contacting a first process offgas stream comprising hydrogen chloride and phosgene with an absorbent and also comprising a computer for closed-loop control of the separation, characterized in that that the computer is set up for the control of the process of the invention.

Alternatively or preferably, the invention further provides a device for separating off gas streams from a phosgenation reaction, comprising
at least one absorption device for contacting a first process offgas stream comprising hydrogen chloride and phosgene with an absorbent Ml, affording a phosgene-depleted second offgas stream having a first phosgene content and a first phosgene solution having a second phosgene content,
an apparatus, associated with the absorption device, for determining the first phosgene content in the second offgas stream and/or an apparatus, associated with the absorption device, for determining the second phosgene content of the first phosgene solution,
an apparatus for altering the absorbent mass flow M1 with which the absorbent is introduced into the absorption device,
and also optionally a computer for closed-loop control of the separation, characterized in that the computer is set up for the control of the process of the invention.

The apparatus used for determining the first phosgene content for the aforementioned devices of the invention can for example be a sampling station or an online analyzer. The sampling station is capable of collecting samples from the offgas stream that can then be analyzed, for example by spectrometric methods, gas chromatography or wet-chemical analysis. Preferably, it is an online analyzer capable of determining the content of phosgene in the offgas stream by IR spectrometry. Preferably, the device of the invention comprises at least one device for offgas post-treatment, wherein the apparatus for determining the first phosgene content in the second offgas stream is arranged between the absorption device and the at least one device for offgas post-treatment. The term “between” is in this context to be understood as meaning between the process steps and not necessarily as a spatial arrangement. The offgas post-treatment device is suitable for removing any residual phosgene from the offgas stream. Such offgas post-treatment devices are known to those skilled in the art. For example, these may be alkaline scrubbers or activated carbon towers. The advantage of arranging the apparatus for determining the first phosgene content upstream of such an offgas post-treatment device is that not only does this permit more direct closed-loop control of the absorption device, but it can also allow post-treatment to be omitted, with the offgas only supplied to the offgas post-treatment device, or said device only activated, when phosgene is detected in the offgas stream.

The apparatus employed for altering the absorbent mass flow M1 for the device of the invention may for example be a control valve, a delivery pump with adjustable delivery rate or else a bypass line with adjustable flow rate. Preferably, the apparatus for altering the absorbent mass flow M1 is a closed-loop control valve capable of restricting the flow of absorbent in the corresponding supply line.

### EXAMPLES

### Comparative Example 1

A solution of 120 kg of phosgene in 300 kg of chlorobenzene was prepared at 0° C. in a 1 m³ stirred-tank reactor with temperature control unit and fitted with a reflux condenser. To the phosgene solution was added, with stirring, a suspension of 50 kg of 1,5-diaminonaphthalene in 150 kg of chlorobenzene. The suspension was stirred for approx. 120 minutes without further cooling prior to the transition from the cold phosgenation phase to the hot phosgenation phase. For this, the reaction mixture was heated and then kept under reflux for about 6 h. The pressure during the reaction was approx. 2.9 bar(a). After the reaction mixture had become clear, which signals the end of the phosgenation reaction, the reactors were first depressurized. The liquid crude product was then run off and the isocyanate isolated in a distillation sequence.

During the production run, the process offgas was supplied to a two-stage absorption column in which it was washed with chlorobenzene in countercurrent. In said run, the process offgas passed first through an isothermal absorption stage at approx. −15° C. and then an adiabatic absorption stage, before exiting the process as offgas. Fresh chlorobenzene was added at −5° C. at the upper end of the adiabatic absorption stage. The mass flow of fresh chlorobenzene to the absorption column was approx. 20 kg/h. After passing through the adiabatic absorption stage, the solution of phosgene in chlorobenzene obtained therein was forwarded as a wash solution to the isothermal absorption stage, with the result that a solution of phosgene in MCB was ultimately withdrawn from the bottom of the two-stage absorption column, and this, after adjusting the phosgene concentration, served as a starting material for further phosgenations. The concentration of the withdrawn phosgene solution decreased in the course of the reaction, which meant that an increasing amount of fresh phosgene needed to be added to adjust the phosgene concentration for further use.

In the transition to the hot phosgenation, the process offgas mass flow from the reactor passed through at a maximum of 70 kg/h and the process offgas consisted of approx. 90% phosgene and approx. 10% hydrogen chloride. The ratio of chlorobenzene to phosgene at the inlet to the absorption column at this point was accordingly approx. 0.3:1 and a hydrogen chloride stream freed of phosgene was obtained at the outlet of the absorption column. Later on in the batchwise process, the overall mass flow of process offgas decreased and the composition shifted toward higher hydrogen chloride contents. Surprisingly, a penetration of phosgene into the absorption column offgas occurred during this phase. 5.2% by volume of phosgene was detected in the offgas stream by IR measurement. The ratio of hydrogen chloride to phosgene at the inlet to the absorption column at this point was approx. 1.3:1.

At the end of the reaction, the process offgas stream ceased. There was one more short-lived increase in the mass flow of process offgas in the collecting line solely when the pressure in the reactors was released, but this was much less pronounced than the first maximum.

Comparative example 1 shows clearly that, with a constant MCB mass flow into the absorption device, problems occur in the operation of said device, these being, firstly, a significant fluctuation in the composition of the phosgene solution obtained and, secondly, periods of phosgene penetration into the offgas of the absorption device. This is the case even though the amount ratios of phosgene to MCB at the inlet to the absorption device corresponded to the amount ratios recommended in the prior art.

### Comparative Example 2

The synthesis from comparative example 1 was repeated under the same reaction conditions. Unlike in comparative example 1, the absorption device was in this case not charged with a constant mass flow of chlorobenzene. Instead, this was varied as a function of the phosgene mass flow in the process offgas, such that the mass flow of fresh hydrogen chloride into the absorption column always corresponded to 1.3 times the mass flow of phosgene into the absorption column, provided that the irrigation density of the absorption column was not below the minimum value. The maximum mass flow of chlorobenzene during the transition from the cold phosgenation to the hot phosgenation, i.e. at the point of greatest phosgene mass flow in the process offgas, was 82 kg/h.

During the hot phosgenation, there was again significant phosgene penetration into the offgas of the absorption device. In this experiment too, more than 5% by volume of phosgene was detected in the offgas. Over the total run time of batch production, the consumption of chlorobenzene was higher than in example 1, with the result that the concentration established in the phosgene solution obtained in the column bottoms was on average lower and it was necessary to add a correspondingly greater amount of phosgene in order to obtain the desired phosgene concentration.

### Example 1 (According to the Invention)

The synthesis from comparative example 1 was repeated in a run in which a closed-loop control unit that controls the mass flow of absorbent had been set up for the operation of the absorption column. For this purpose, the phosgene content in the off gas from the absorption device was tracked by online IR measurement. The target value for phosgene content was set at 0.05% by volume and the deviation of the measured value from this target value acted, via a closed-loop control circuit, ultimately on a control valve in the fresh chlorobenzene supply line, so as to adjust the mass flow of chlorobenzene to the absorption column accordingly and to keep the deviation as small as possible.

Over the entire production time of the batch, this made it possible to minimize the use of chlorobenzene and ensure that the offgas from the absorption column was only minimally contaminated with phosgene. The observed phosgene content fluctuated between 0.03% by volume and 0.08% by volume. A further post-treatment to destroy residual phosgene was thus readily possible. The concentration of the phosgene solution in the bottoms fluctuated, but on average assumed the maximum possible value that was achievable without penetration of larger amounts of phosgene into the offgas.

### Example 2 (According to the Invention)

The synthesis from example 1 was repeated employing a different closed-loop control concept. The primary focus of said closed-loop control was on a constant phosgene content in the phosgene solution obtained in the bottoms of the absorption column (closed-loop control of the bottoms concentration). This was done by determining said concentration as a closed-loop control variable and comparing it against the desired target value. This closed-loop control difference was evaluated and the mass flow of MCB to the absorption column automatically adjusted in such a way as to minimize the difference. In addition, an override concept was implemented. For this, a threshold value of 0.05% by volume was set in this case for the phosgene content in the offgas downstream of the absorption column; if this was exceeded, the closed-loop control of the bottoms concentration was deactivated and the MCB mass flow to the absorption column increased by a predefined value until the phosgene content of the offgas had fallen below the threshold value again.

This operating mode resulted in a very constant phosgene content of approx. 55% by weight in the phosgene solution obtained in the bottoms. During the hot phosgenation, the override mode was activated for a few minutes; the consequent increase in mass flow of absorbent led to a decrease in the phosgene content that was compensated by adding more phosgene to the phosgene solution. In contrast to comparative example 2, the override allowed the phosgene content in the offgas to be kept below 0.2% by volume.

### Example 3 (According to the Invention)

In contrast to the examples and comparative examples described above, the reaction in example 3 was carried out in two identically equipped reactors arranged in parallel. The total batch size was divided in half between the two reactors and the reaction was carried out in the two reactors with a start time 3 h apart. The process offgas was combined in a collecting line and supplied to the absorption device, which was otherwise operated with a closed-loop control unit as in example 1. Since the maximum mass flows of process offgas from the two reactors did not occur at the same time, the maximum gas loading of the absorption column was also lower. The observed phosgene content in the offgas fluctuated between 0.04% by volume and 0.07% by volume. cm 1. A process for producing an isocyanate, comprising the steps of:
A) reacting at least one amine or salt thereof with phosgene, producing a liquid reaction product comprising the isocyanate and also a first gaseous process offgas stream comprising hydrogen chloride and phosgene,
B) separating the gaseous process offgas stream in at least one absorption device, in which the first process offgas stream is contacted with an absorbent, producing a phosgene-depleted second offgas stream having a first phosgene content and a first phosgene solution having a second phosgene content,
C) optionally mixing the first phosgene solution obtained in step B) with additional phosgene or solvent to obtain a conditioned second phosgene solution,
D) optionally using the first phosgene solution from step B) or the conditioned second phosgene solution from step C) for phosgenation of an amine,
E) optionally isolating the isocyanate by distillative separation of the liquid reaction product obtained in step A),
   wherein
the first phosgene content of the phosgene-depleted second offgas stream obtained in step B) is compared against a first target value for first phosgene content or the second phosgene content of the first phosgene solution obtained in step B) is determined and compared against a predefined second target value for said second phosgene content,
wherein the absorbent having a mass flow M1 is introduced into the absorption device and this mass flow M1 is under closed-loop control, and
wherein the reference variable for the mass flow M1 is altered when the first phosgene content or the second phosgene content deviate from their respective target value. cm 2. The process of claim 1, wherein the target value for the first phosgene content is in the range from 0.00% by volume to 2.00% by volume, preferably in the range from 0.01% by volume to 1.00% by volume, more preferably in the range from 0.02% by volume to 0.50% by volume, and most preferably in the range from 0.02% by volume to 0.2% by volume.

3. The process of claim 1, wherein the target value for the second phosgene content is in the range from 20% by weight to 70% by weight.

4. The process of claim 1, wherein, in normal operation, the second phosgene content is controlled by adjusting the mass flow M1 to the predefined target value and, if the threshold value for the first phosgene content is exceeded, there is a deviation from normal operation, and the mass flow M1 is increased.

5. The process of claim 1, wherein the at least one amine is a diamine.

6. The process of claim 1, wherein the reaction of the at least one amine or salt thereof with phosgene is carried out in the presence of a solvent.

7. The process of claim 1, wherein the reaction of the at least one amine or salt thereof with phosgene is carried out in the condensed phase.

8. The process of claim 1, wherein the reaction of the at least one amine or salt thereof with phosgene is carried out in a batchwise manner.

9. The process of claim 1, wherein the reaction in step A) is a base phosgenation of at least one amine with phosgene.

10. The process of claim 8, wherein the reaction in step A) is carried out in at least 2 reactors arranged in parallel, where at least one reactor is operated asynchronously to at least one of the other reactors.

11. The process claim 1, wherein chlorobenzene, o-dichlorobenzene or mixtures of these two solvents is used as the absorbent step B).

12. The process of claim 6, wherein a solvent is employed as the absorbent in step B), which is the solvent that is also present in the reaction of the at least one amine or salt thereof.

13. The process of claim 1, wherein the absorption device comprises a gas scrubber selected from the group consisting of packed columns with a random packing, packed columns with a structured packing, and tray columns, preferably consisting of packed columns with a random packing and packed columns with a structured packing.

14. A device for separating offgas streams from a phosgenation reaction, comprising
at least one absorption device for contacting a first process offgas stream comprising hydrogen chloride and phosgene with an absorbent M1, producing a phosgene-depleted second offgas stream having a first phosgene content and a first phosgene solution having a second phosgene content,
an apparatus, associated with the absorption device, for determining the first phosgene content in the second offgas stream or an apparatus, associated with the absorption device, for determining the second phosgene content of the first phosgene solution,
an apparatus for altering the absorbent mass flow M1 with which the absorbent is introduced into the absorption device,
and a computer for closed-loop control of the separation, wherein the computer is set up for the control of the process of claim 1.

15. The device of claim 14, wherein the device comprises at least one device for offgas post-treatment, wherein the apparatus for determining the first phosgene content in the second offgas stream is arranged between the absorption device and the at least one device for offgas post-treatment.

16. The process of claim 5, wherein the at least one amine is a diamine selected from the group consisting of 1,5-diaminonaphthalene (NDA), 1,4-diaminobenzene (pPDA), 1,5-diaminopentane (PDA), bis(p-aminocyclohexyl)methane (PACM), hexahydrotolylenediamine (H6TDA), 2,4-hexahydrotolylenediamine and 2,6-hexahydrotolylenediamine, 1,3-bis(aminomethyl)benzene (m-XDA), bis(aminomethyl)norbornane (NBDA), and mixtures thereof.

17. The process of claim 3, wherein the target value for the second phosgene content is in the range from 30% by weight to 68% by weight.

18. The process of claim 3, wherein the target value for the second phosgene content is in the range from 42% by weight to 66% by weight.

19. The process of claim 2, wherein the target value for the first phosgene content is in the range from 0.01% by volume to 1.00% by volume.

20. The process of claim 2, wherein the target value for the first phosgene content is in the range from 0.02% by volume to 0.2% by volume.
